(19) United States
(12) Patent Application Publication
(10) Pub. No.: US 2021/0177523 A1
(43) Pub. Date: Jun. 17, 2021

(54) LOW PROFILE ENT PROBE WITH DISTAL SHAFT LENGTH AND RIGIDITY ADJUSTMENT

(71) Applicant: Acclarent, Inc., Irvine, CA (US)

(72) Inventors: Babak Ebrahimi, Irvine, CA (US); Ehsan Shameli, Irvine, CA (US); Fatemeh Akbarian, Rancho Palos Verdes, CA (US); Roozbeh Borjian, Irvine, CA (US); Marc Dean, Fort Worth, TX (US)

(21) Appl. No.: 17/099,874

(22) Filed: Nov. 17, 2020

Related U.S. Application Data
(60) Provisional application No. 62/948,547, filed on Dec. 16, 2019.

(51) Int. Cl. A61B 34/20; A61B 1/227; A61B 1/273; A61B 1/233; A61B 1/005; A61B 1/00

(52) U.S. Cl. CPC A61B 34/20; A61B 1/227; A61B 1/273; A61B 2034/2059; A61B 1/0052; A61B 1/00158; A61B 1/233

(57) ABSTRACT

An apparatus includes a handle, an elongate shaft assembly, an adjustment feature, and a position sensor. The shaft assembly includes an outer tube and an inner shaft. The outer tube is slidably disposed within an elongate body of the handle. The outer tube extends distally from the elongate body to an outer tube distal end. The inner shaft is coaxially disposed within the outer tube and extends distally through the outer tube end to an inner shaft distal tip. The inner shaft is sized and configured to pass through passageways within an ear, nose, or throat of a patient. The adjustment feature is slidably disposed relative to the body of the handle and is configured to translate at least the outer tube relative to the handle. The position sensor is configured to generate signals indicating a real-time position of the distal tip of the inner shaft.

### PRIORITY

This application claims the benefit of U.S. Provisional Pat. App. No. 62/948,547, filed Dec. 16, 2019, entitled “Low Profile ENT Probe with Distal Shaft Length and Rigidity Adjustment,” the disclosure of which is incorporated by reference herein.

### BACKGROUND

Image-guided surgery (IGS) is a technique where a computer is used to obtain a real-time correlation of the location of an instrument that has been inserted into a patient's body to a set of preoperatively obtained images (e.g., a CT or MRI scan, 3-D map, etc.), such that the computer system may superimpose the current location of the instrument on the preoperatively obtained images. An example of an electromagnetic IGS navigation systems that may be used in IGS procedures is the CARTO® 3 System by Biosense-Webster, Inc., of Irvine, Calif. In some IGS procedures, a digital tomographic scan (e.g., CT or MRI, 3-D map, etc.) of the operative field is obtained prior to surgery. A specially programmed computer is then used to convert the digital tomographic scan data into a digital map. During surgery, special instruments having sensors (e.g., electromagnetic coils that emit electromagnetic fields and/or are responsive to externally generated electromagnetic fields) are used to perform the procedure while the sensors send data to the computer indicating the current position of each surgical instrument. The computer correlates the data it receives from the sensors with the digital map that was created from the preoperative tomographic scan. The tomographic scan images are displayed on a video monitor along with an indicator (e.g., crosshairs or an illuminated dot, etc.) showing the real-time position of each surgical instrument relative to the anatomical structures shown in the scan images. The surgeon is thus able to know the precise position of each sensor-equipped instrument by viewing the video monitor even if the surgeon is unable to directly visualize the instrument itself at its current location within the body.

When ENT procedures are performed, the use of image guidance systems allows the surgeon to achieve more precise movement and positioning of the surgical instruments than can be achieved by viewing through an endoscope alone. This is so because a typical endoscopic image is a spatially limited, 2-dimensional, line-of-sight view. The use of image guidance systems provides a real time, 3-dimensional view of all of the anatomy surrounding the operative field, not just that which is actually visible in the spatially limited, 2-dimensional, direct line-of-sight endoscopic view. As a result, image guidance systems may be particularly useful during ENT procedures where a section and/or irrigation source may be desirable, especially in cases where normal anatomical landmarks are not present or are difficult to visualize endoscopically.

In some instances, it may be desirable to access anatomies with a surgical instrument to facilitate operating within or adjacent to an anatomical passageway of a patient, such as performing an incision of mucosa, removal of bone, or dilation of an anatomical passageway. Such operations may occur within anatomical passageways such as ostia of paranasal sinuses (e.g., to treat sinusitis), the larynx, the Eustachian tube, or other passageways within the ear, nose, or throat, etc. In addition to the above described operations, or similar operations, it may be desirable to probe within or adjacent to an anatomical passageway before, during, or after the above described operations, or similar operations. One method of probing within or adjacent to an anatomical passageway of a patient involves obtaining a probe that includes an elongate shaft assembly having a fixed working length and rigidity that extends from the handle to a distal end. An operator may then insert the distal end of the elongate shaft assembly within the nostril or mouth of a patient toward a targeted anatomy. With the distal end of the elongate shaft assembly inserted within the patient, the operator may manipulate the probe in order to manipulate or explore the targeted anatomies. Having a probe with a shaft assembly having an adjustable working length, and an adjustable rigidity, may be beneficial for multiple purposes as will be apparent to those skilled in the art.

It may be desirable to provide features that further facilitate the use of an IGS navigation system and associated components in ENT procedures and other medical procedures. While several systems and methods have been made and used in surgical procedures, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1 depicts a schematic view of an exemplary surgery navigation system being used on a patient seated in an exemplary medical procedure chair;

FIG. 2 depicts a perspective view of an exemplary probe having an outer tube of a shaft assembly with an adjustable length;

FIG. 3A depicts a perspective view of the probe of FIG. 2 with the outer tube in the extended position;

FIG. 3B depicts a perspective view of the probe of FIG. 2 with the outer tube in the retracted position;

FIG. 4 depicts an enlarged perspective view of the probe of FIG. 2 with the handle shown in phantom for additional clarity to show an adjustment feature;

FIG. 5A depicts another exemplary probe with a shaft assembly in an extended position;

FIG. 5B depicts the probe of FIG. 5A with the shaft assembly in a retracted position;

FIG. 6A depicts yet another exemplary probe with an inner shaft and an outer tube of a shaft assembly both in extended positions;

FIG. 6B depicts the probe of FIG. 6A with the inner shaft in the extended position and the outer tube in a retracted position;

FIG. 6C depicts the probe of FIG. 6A with the inner shaft in a retracted position and the outer tube in the extended position; and

FIG. 6D depicts the probe of FIG. 6A with both the inner shaft and outer tube in the retracted positions.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top”, “bottom”, “left”, and “right” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

I. Exemplary Image Guided Surgery Navigation System

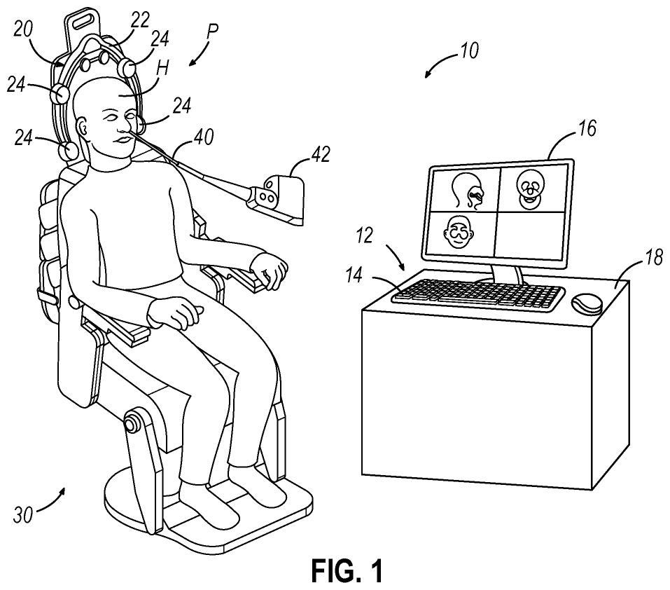

When performing a medical procedure within a head (H) of a patient (P), it may be desirable to have information regarding the position of an instrument within the head (H) of the patient (P), particularly when the instrument is in a location where it is difficult or impossible to obtain an endoscopic view of a working element of the instrument within the head (H) of the patient (P). FIG. 1 shows an exemplary IGS navigation system (10) enabling an ENT procedure to be performed using image guidance. In addition to or in lieu of having the components and operability described herein IGS navigation system (10) may be constructed and operable in accordance with at least some of the teachings of U.S. Pat. No. 7,720,521, entitled “Methods and Devices for Performing Procedures within the Ear, Nose, Throat and Paranasal Sinuses,” issued May 18, 2010, the disclosure of which is incorporated by reference herein; and U.S. Pat. Pub. No. 2014/0364725, entitled “Systems and Methods for Performing Image Guided Procedures within the Ear, Nose, Throat and Paranasal Sinuses,” published Dec. 11, 2014, now abandoned, the disclosure of which is incorporated by reference herein.

IGS navigation system (10) of the present example comprises a field generator assembly (20), which comprises set of magnetic field generators (24) that are integrated into a horseshoe-shaped frame (22). Field generators (24) are operable to generate alternating magnetic fields of different frequencies around the head (H) of the patient (P). A navigation guidewire (40) is inserted into the head (H) of the patient (P) in this example. Navigation guidewire (40) may be a standalone device or may be positioned on an end effector or other location of a medical instrument such as a surgical cutting instrument or dilation instrument. In the present example, frame (22) is mounted to a chair (30), with the patient (P) being seated in the chair (30) such that frame (22) is located adjacent to the head (H) of the patient (P). By way of example only, chair (30) and/or field generator assembly (20) may be configured and operable in accordance with at least some of the teachings of U.S. Patent Pub. No. 2018/0310886, entitled “Apparatus to Secure Field Generating Device to Chair,” published on Nov. 1, 2018, now abandoned, the disclosure of which is incorporated by reference herein. Navigation guidewire (40) that is responsive to positioning within the alternating magnetic fields generated by field generators (24). Coupling unit (42) may provide wired or wireless communication of data and other signals.

In the present example, the sensor of navigation guidewire (40) comprises at least one coil at the distal end of navigation guidewire (40). A coupling unit (42) is secured to the proximal end of navigation guidewire (40) and is configured to provide communication of data and other signals between processor (12) and navigation guidewire (40). Processor (12) of the present example is mounted in a console (18), which comprises operating controls (14) that include a keypad and/or a pointing device such as a mouse or trackball. When such a coil is positioned within an alternating electromagnetic field generated by field generators (24), the alternating magnetic field may generate electrical current in the coil, and this electrical current may be communicated along the electrical conduit(s) in navigation guidewire (40) and further to processor (12) via coupling unit (42). This phenomenon may enable IGS navigation system (10) to determine the location of the distal end of navigation guidewire (40) or other medical instrument (e.g., dilation instrument, surgical cutting instrument, etc.) within a three-dimensional space (i.e., within the head (H) of the patient (P), etc.). To accomplish this, processor (12) executes an algorithm to calculate location coordinates of the distal end of navigation guidewire (40) from the position related signals of the coil(s) in navigation guidewire (40). While the position sensor is located in guidewire (40) in this example, such a position sensor may be integrated into various other kinds of instruments, including those described in greater detail below.

IGS navigation system (10) of the present example further comprises a processor (12), which uses software stored in a memory of processor (12) to calibrate and operate IGS navigation system (10). Such operation includes driving field generators (24) to generate alternating electromagnetic fields, processing signals from navigation guidewire (40) to determine the location of a sensor in navigation guidewire (40) within the head (H) of the patient (P) and driving display screen (16). In some implementations, operation may also include monitoring and enforcement of one or more safety features or functions of IGS navigation system (10). Processor (12) is further operable to provide video in real time via display screen (16), showing the position of the distal end of navigation guidewire (40) in relation to a video camera image of the patient's head (H), a CT scan image of the patient's head (H), and/or a computer-generated three-dimensional model of the anatomy within and adjacent to the patient's nasal cavity. Display screen (16) may display such images simultaneously and/or superimposed on each other during the surgical procedure. Such displayed images may also include graphical representations of instruments that are inserted in the patient's head (H), such as navigation guidewire (40), such that the operator may view the virtual rendering of the instrument at its actual location in real time. By way of example only, display screen (16) may provide images in accordance with at least some of the teachings of U.S. Pat. No. 10,463,242 entitled “Guidewire Navigation for Sinuplasty,” issued as on Nov. 5, 2019, the disclosure of which is incorporated by reference herein.

The images provided through display screen (16) may help guide the operator in maneuvering and otherwise manipulating instruments within the patient's head (H) when such instruments incorporate navigation guidewire (40). It should also be understood that other components of a surgical instrument and other kinds of surgical instruments, as described below, may incorporate a sensor like the sensor of navigation guidewire (40).

II. Exemplary ENT Probe with Shaft Assembly Having Adjustable Rigidity

In some scenarios, an operator may use a probe to explore cavities, confirm the location or state of anatomical landmarks (such as sinus ostia), and manipulate tissue within the head (H) of the patient (P). The operator may need to access multiple different cavities and anatomical passageways within the head, nose and throat. A probe having a selectable rigidity may be desirable to the operator. A probe having a flexible elongate shaft assembly may be desirable to aid the operator in accessing remote cavities within the head (H) of the patient (P); but when manipulating tissue or exploring certain regions within the head (H) of the patient (P), a rigid elongate shaft assembly may be alternately desirable. It may therefore be desirable to provide a probe with a shaft assembly that provides user adjustment of flexibility in the shaft assembly. In the event that a probe has a position sensor like guidewire (40) described above, the probe may be used in conjunction with an IGS navigation system (10) to assist in mapping regions and routes within the head (H) of the patient (P), further correlating spaces within the head (H) in real time with those same spaces as represented in preoperatively obtained images that are stored in IGS navigation system (10).

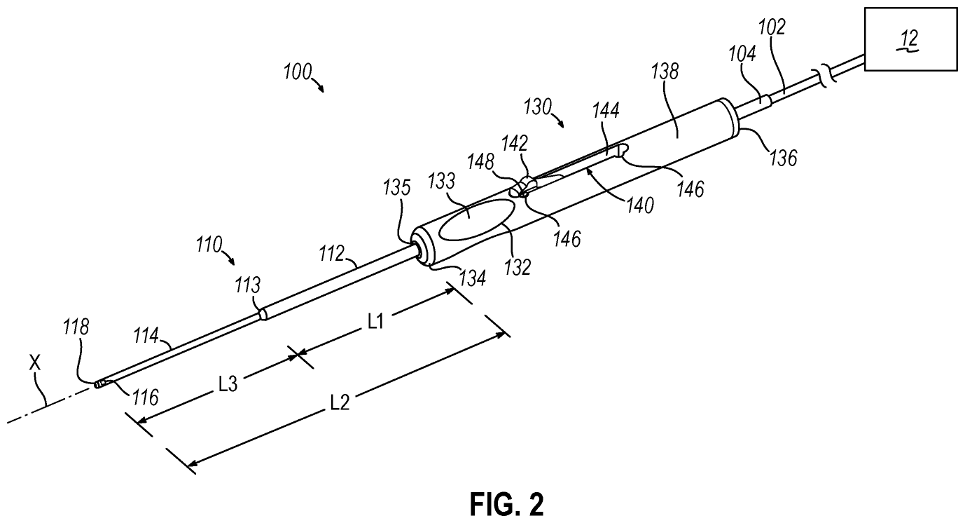

FIG. 2 shows an exemplary probe (100) that may be readily incorporated into IGS navigation system (10) described above in order to obtain a reference to help navigate the probe (100) within or adjacent to anatomical passageways of a patient. The probe (100) comprises a handle (130), a shaft assembly (110), and a position sensor (116).

The handle (130) includes an elongate body (138) that is tubular in shape and includes a bore (135), a proximal end (136) and a distal end (134). The handle (130) is dimensioned to be grasped by the operator such that the operator may control the probe (100) using just one single hand. The proximal end (136) attaches to the communication cable (102) via the cable guard (104). The elongate body (138) further includes an adjustment feature (140) and a grip (132) between ends (134, 136).

The grip (132) is positioned distal to the proximal end (136) and proximal to the distal end (134). The grip (132) has an oval section (133) that is inset from a surface of the elongate body (138) to facilitate gripping of the elongate body with fingers and a thumb. In the current example, the grip (132) has three oval sections (133). The grip may be configured with one or more of these oval sections (133). The grip (132) may be configured from a material that increases the tack of the grip such as silicone or rubber. Alternatively, grip (132) may take any other suitable form.

The adjustment feature (140) of the present example is in the form of a slider that is disposed in an elongate aperture (144). The elongate aperture (144) is located on the surface of the elongate body (138). The adjustment feature is in a position that is accessible by a thumb or finger of the operator when the operator is grasping the grip (132). The elongate aperture (144) is a channel that runs longitudinally through the elongate body (138). In the current example, the adjustment feature (140) is located proximate to the grip (132) and generally equidistant between the proximal end (136) and the distal end (134).

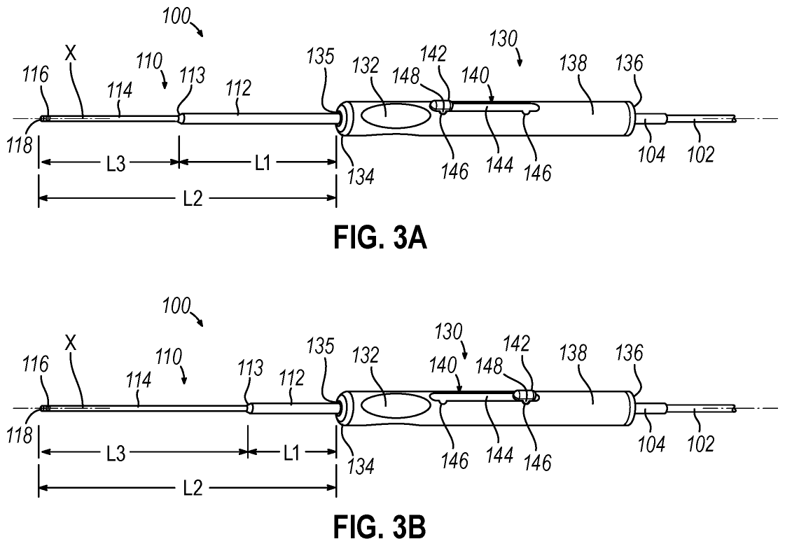

The shaft assembly (110) extends distally from the distal end (134) of the handle (130) to the distal tip (118). The shaft assembly (110) includes an outer tube (112), an inner shaft (114), and a position sensor (116). The outer tube (112) is a hollow tube that is coaxially located within the elongate body (138) and extends distally an effective outer tube length (L1) from the distal end (134) of the handle (130) to the outer tube end (113), as shown in FIGS. 3A-3B. The outer tube (112) is configured to be in mechanical communication with the adjustment feature (140). The outer tube (112) is constructed of a rigid material such as hard plastic, aluminum, or stainless steel.

The inner shaft (114) is coaxially located within the outer tube (112) and extends distally from within the elongate body (138) through a distal end of the outer tube (112) to a distal tip (118). The distal tip (118) is an effective working length (L2) from the handle distal end (134), as also shown in FIGS. 3A-3B. This effective working length (L2) determines the working length of the probe (100). In the present example, this effective length (L2) is fixed at all times, regardless of the longitudinal position of adjustment feature (140) relative to elongate body (138). The inner shaft (114) is constructed of a soft, flexible material such as plastic or rubber.

The position sensor (116) is located at or near the distal tip (118) of the inner shaft (114). The position sensor (116) communicates with the processor (12) via a communication cable (102). The communication cable (102) includes a cable guard (104) that is configured to serve as a strain relief and prevent the communication cable (102) from chafing or breaking. The cable guard (104) coaxially encompasses the communication cable (102) and keeps the communication cable (102) aligned with a proximal end of the handle (130).

Additionally, communication cable (102) is configured to selectively couple with console (18) (see FIG. 1) with a connector (not shown) in order to communicate with processor (12) (see FIG. 1). As described in greater detail below, position sensor (116) may be utilized with processor (12) to determine the location of the position sensor (116) within the head (H) of patient (P) relative to field generators (24) (see FIG. 1) during exemplary use.

The communication cable (102) is configured to provide a conduit for communication between position sensor (116) and processor (12) during exemplary use. Therefore, communication cable (102) may directly connect with console (18) such that position sensor (116) is in wired communication with processor (12) via communication cable (102). Alternatively, communication cable (102) may connect position sensor (116) with a wireless communication device that is in wireless communication with console (18), similar to how coupling unit (42) (see FIG. 1) establishes wireless communication between navigation guidewire (40) (see FIG. 1) and console (18). As another merely illustrative example, probe (100) may integrate a wireless communication module that is operable to communicate wirelessly with processor (12), such that communication cable (102) may be omitted.

By way of example only, position sensor (116) may comprise one or more coils of wire wrapped around one or more respective axes. Thus, like the position sensor of navigation guidewire (40) described above, position sensor (116) may generate electrical currents/signals in response to the alternating magnetic fields generated by field generators (24) during exemplary use. Such signals generated by position sensor (116) may be sent to processor (12) as described above. Because position sensor (116) is fixed relative distal tip (118), the signals indicating the position of position sensor (116) within three-dimensional space may indicate the position of distal tip (118) within the head (H) of the patient (P) in accordance with the description above. Therefore, signals generated by position sensor (116) may allow processor (12) to determine the real-time three-dimensional location and orientation of distal tip (118) within the head (H) of patient (P) relative to the plurality of field generators (24) fixed to horseshoe-shaped frames (22) (see FIG. 1) and chair (30) (see FIG. 1).

FIG. 3A-3B shows the adjustment feature (140) and the outer shaft (112) in an extended, distal position (FIG. 3A) and a retracted, proximal position (FIG. 3B). With outer shaft (112) the distal position, the shaft assembly (110) is rigid relative to shaft assembly (110) with outer shaft (112) in the proximal position (see FIG. 3B). In use, the rigidity of shaft assembly (110) is selectively determined by adjusting the adjustment feature (140). The adjustment feature (140) may be pushed distally or proximally by a thumb or a finger of the operator. The adjustment feature (140) is operatively attached to the outer shaft (112). When the adjustment feature (140) is transitioned proximally, the adjustment feature (140) engages the outer tube (112) and the outer tube (112) retracts proximally, reducing the effective outer tube length (L1). The effective outer tube length (L1) is the length from the bore (135) to the outer tube end (113).

With outer tube (112) in the extended position, the effective outer tube length (L1) is longer than the effective outer tube length (L1) when outer tube (112) is in the retracted position (see FIG. 3B). A reduction in the effective outer tube length (L1) increases the effective inner shaft length (L3). The effective inner shaft length (L3) is the length from the outer tube end (113) to the distal tip (118). A longer effective inner shaft length (L3) makes shaft assembly (110) effectively more flexible. The effective working length (L2) of shaft assembly (110) is held constant. The effective working length (L2) is the distance from the distal tip (118) to the bore (135). The shorter the effective inner shaft length (L3), the more support the rigid outer tube (112) gives the flexible inner shaft (114). The more support that the flexible inner shaft (114) has, the more rigid the shaft assembly (110) is as a whole. Thus, with outer tube (112) the distal position (FIG. 3A), the shaft assembly (110) is effectively more rigid than shaft assembly (110) is when outer tube (112) is in the proximal position (FIG. 3B).

FIG. 3B shows the adjustment feature (140), and outer tube (112) transitioned into the retracted, proximal position. When outer tube (112) is in the retracted position, outer tube (112) exposes more of flexible inner shaft (114). Inner shaft (114) bends more easily with reduced support from the outer tube (112). In order to transition from the extended position to the retracted position, the adjustment feature (140) transitions the outer tube (112) towards the bore (135), reducing the effective outer tube length (L1). The effective working length (L2) does not change but the effective inner shaft length (L3) increases. The increase in the effective length of the effective inner shaft length (L3) makes the shaft assembly (110) more flexible in relation to shaft assembly (110) when outer tube (112) is in the extended position (see FIG. 3A).

In some versions, adjustment feature (140) may only be transitioned between a proximal-most position (FIG. 3B) and a distal-most position (FIG. 3A). In some other versions, the adjustment feature (140) may be placed anywhere between the distal-most position (see FIG. 3A) and the proximal-most position (FIG. 3B) to obtain a desired rigidity of the shaft assembly (110). The adjustment feature (140) may thus have an infinite amount of adjustment between the distal-most position and the proximal-most position. The effective outer tube length (L1) has a direct relationship to the longitudinal position of the adjustment feature (140). For example, if the adjustment feature (140) is translated 2 cm proximally, the outer tube (112) is translated 2 cm proximally. However, in other examples the adjustment feature (140) may use a gearing or another feature to compound the translation of the effective outer tube length (L1). For example, the adjustment feature (140) may be translated 1 cm proximally and the outer tube (112) would be translated 2 cm proximally.

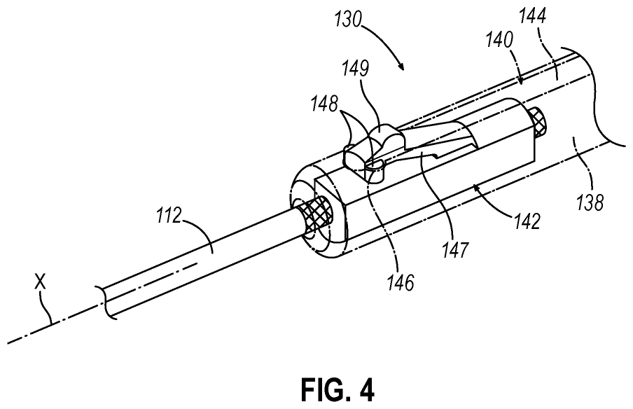

FIG. 4 shows the adjustment feature (140) of probe (100) (see FIG. 2) in a set position. In the set position, the adjustment feature (140) temporarily prohibits the longitudinal movement of adjustment feature (140). The adjustment feature (140) includes a slider body (142), a slider detent (148), and a slider arm (149). The slider body (142) is slidably disposed within the elongate body (138). The slider body (142) is operatively attached to the outer tube (112). The slider body (142) may be press fit, glued, welded, or otherwise secured to outer tube (112). The slider detent (148) is operatively attached to the slider arm (149). The slider detent (148) may be constructed of a shape that mates with a shape of the handle detent (146). The handle detent (146) is a cutaway of the sidewall of the elongate aperture (144) in the present example. In the current example, the slider and handle detents (148, 146) have a circular shape. In other versions, the slider and handle detents (148,146) may be triangular, a square, rectangular, or otherwise shaped. The slider and handle detents (148,146) may be different shapes so long as the interaction between the detents (148,146) inhibit inadvertent movement of the adjustment feature (140) when in the set position.

A plurality of handle detents (146) may be located along the elongate aperture (144). The handle detents (146) are transverse to the elongate aperture (144) and may be arranged in pairs along both edges of the elongate aperture (144) or arranged on one edge of the elongate aperture (144). In other versions, the slider detent (148) may have a cutaway portion, and the handle detent (146) may have an embossed portion.

The slider arm (147) obliquely extends distally away from the slider body (142) to the slider detent (146) relative to the axis (X) that coaxially extends along the length of the elongate body (138). The slider arm (147) has a nub (149) and is resiliently biased toward the set position. The nub (149) protrudes from the distal end of the slider arm (147) and is configured to allow the operator to actuate the slider arm (149). The slider arm (149) may be biased by a spring (not shown) that exhibits a force transverse to the elongate handle (130). In the set position, the slider arm (147) resiliently biases the slider detent (148) into the handle detent (146). The interaction between the two detents (148,146) keeps the adjustment feature (140) from inadvertently being moved longitudinally. There may be various handle detents (146) along the elongate aperture (144). In the current example there are two handle detents (146). A first handle detent (146) is located at the most distal position within the elongate aperture (144) and a second handle detent (146) is located at the most proximal position. However, the handle detents (146) may be located along the elongate aperture (144) in any location that may be desirable (e.g., a set interval such as by distance or a fractional division). An arrangement by distance may be set out by imperial units or metric units. A fractional division may locate the handle detents (146) on the elongate aperture (144) at halves, quarters, or eighths.

In operation, the operator may transition the adjustment feature from the set position to a mobile position by grasping grip (132) with a single hand and depressing the nub (149) with a thumb or finger. The force on the nub (149) depresses the slider arm (147) and uncouples the slider detent (148) from the handle detent (146). The slider arm (147) moves in an arcuate manner toward the axis (X). In some examples, the slider arm (147) is pressed against the slider body (147), in other examples the slider arm (147) comes in close proximity to the slider body (147) but does not engage the slider body (142). Once in the mobile position, the operator may translate the adjustment feature (140) proximally or distally to any desired position along the elongate aperture (144). Once the operator moves the adjustment feature to the desired position and aligns the slider detent (148) with the desired handle detent (148) the operator releases the nub (149). The resiliently biased slider arm (149) angularly transitions away from the longitudinal axis, raising the slider detent (148), which engages the handle detent (146). The detents (146,148) lock the adjustment feature (140) in the desired location.

III. Exemplary ENT Probe with Shaft Assembly Having Adjustable Working Length

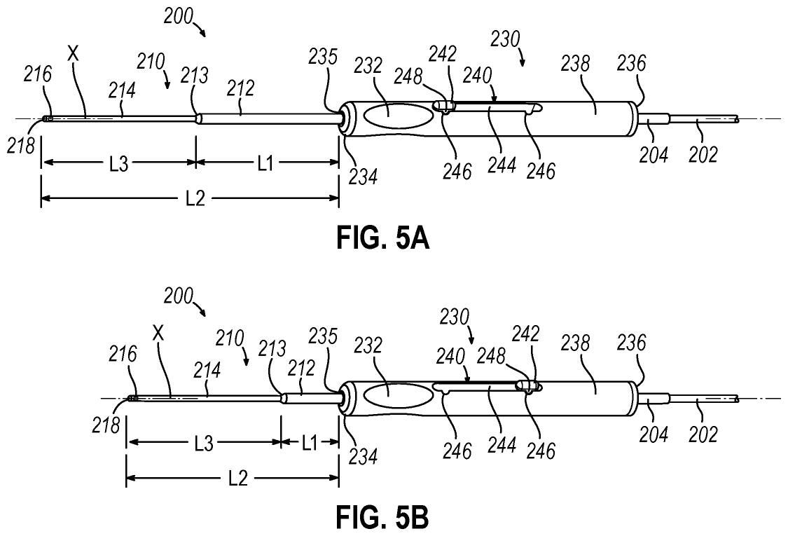

As noted above, an operator may wish to use a probe to explore cavities and manipulate tissue in various locations within the head (H) of the patient (P). To facilitate exploration and manipulation at different depths within various locations within the head (H) of the patient (P), it may be desirable to provide a probe with a shaft assembly that has an adjustable working length. To that end, FIGS. 5A-5B show an exemplary probe (200) with a shaft assembly (210) having an adjustable effective working length (L2). The effective working length (L2) is the distance from the handle distal end (234) to the distal tip (218). It should be understood that probe (200) is substantially similar to probe (100) unless otherwise described herein. Like probe (100), probe (200) has an elongate handle (230), an adjustment feature (240), shaft assembly (210), and a position sensor (216). Both probes (100, 200) may be used to perform operations within the cavities of the ear, nose, and throat. Probe (200) has an elongate housing (240) configured to be grasped by the operator at a grip (232), which is similar to grip (132). Probe (200) also has an adjustment feature (240) that is operable to adjust the effective working length (L2) of shaft assembly (210). The adjustment feature (240) of this example also includes a slider detent (248) that corresponds with a handle detent (246); similar to slider detent (148) and handle detent (146), respectively.

Probes (100, 200) differ in how the adjustment features (140, 240) manipulates the corresponding shaft assembly (110, 210). As described above, adjustment feature (140) changes the effectively rigidity of the shaft assembly (110) by effectively shortening the effective outer tube length (L1) without changing the effective working length (L2). Like probe (100), outer tube length (L1) is the distance from handle distal end (234) to the outer tube end (213). In the example of probe (200), the adjustment feature (240) changes the effective working length (L2) and the effective outer tube length (L1), but shaft assembly (210) has a constant effective rigidity because the effective inner shaft length (L3) does not change. The effective inner shaft length (L3) is the distance from the outer tube end (213) to the distal tip (218). The adjustment feature (240) may be operatively connected to both the outer tube (212) and the inner shaft (214).

FIG. 5A shows probe (200) with shaft assembly (210) in the extended, distal position. With shaft assembly (210) in the distal position the adjustment feature (240) is distally located within elongate aperture (244) of handle (230). Shaft assembly (210) transitions from the distal position to the proximal position similarly to outer tube (112). An operator uncouples the slider detent (248) from the handle detent (246) with a finger or a thumb and translates the slider body (242) proximally along the elongate aperture (244) until the slider body (242) is located proximally (see FIG. 5B). In the current example, the effective working length (L2) is reduced by the distance the adjustment feature (240) is translated proximally. The inner shaft (214) and the outer tube (212) translate together, coaxially through bore (235). The effective inner shaft length (L3) remains the same length in all positions that the adjustment feature (240) may obtain. Since the effective inner shaft length (L3) determines the extent to which shaft assembly (210) is effectively rigid or flexible, the effective rigidity of shaft assembly (210) does not change when adjustment feature (240) is transitioned distally or proximally relative to body (238) of handle (230).

IV. Exemplary ENT Probe with Shaft Assembly Having Independently Adjustable Rigidity and Working Length

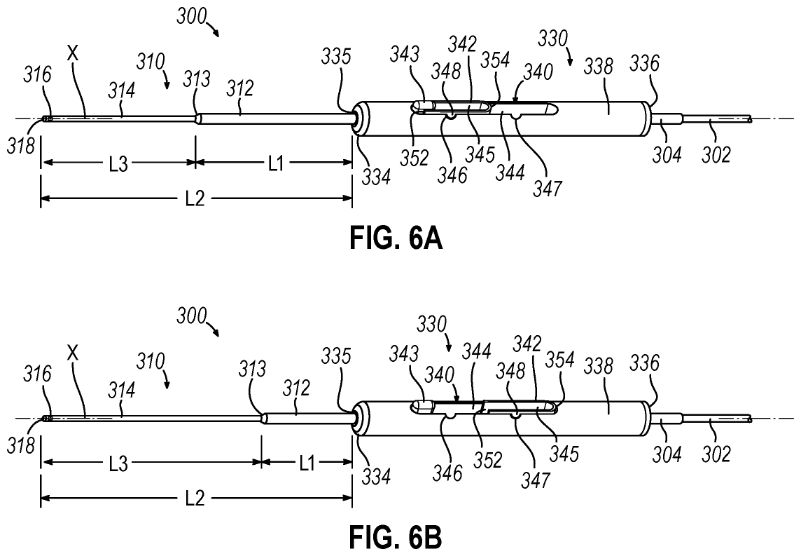

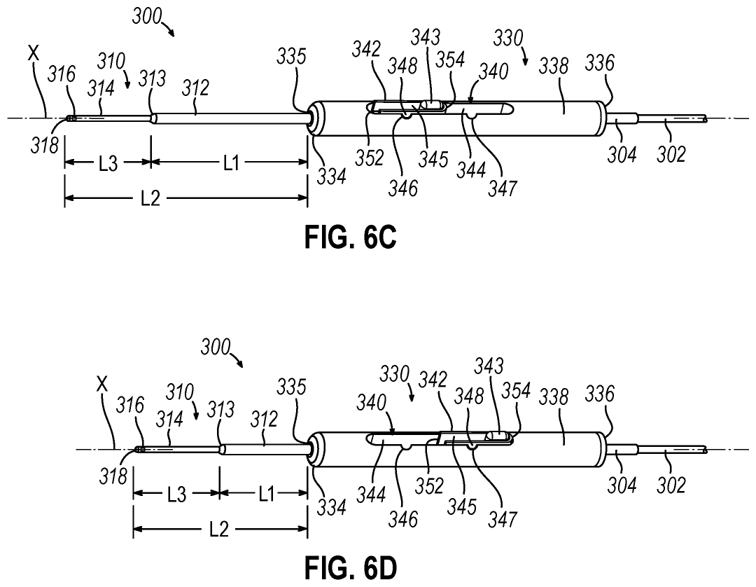

As noted above, it may be desirable to provide a probe with a shaft assembly where the effective rigidity of the shaft assembly can be selectively adjusted by an operator. As also noted above, it may be desirable to provide a probe with a shaft assembly that has an adjustable working length. While the examples provided above include probes (100, 200) that include such functionality separately, it may be further desirable to provide a probe that includes all such functionality. Such a probe may further facilitate exploration and manipulation within various cavities within the ear, nose, and throat of the patient (P). To that end, FIGS. 6A-6D show an exemplary probe (300) with a shaft assembly (310) that is configured to have an adjustable effective working length (L2), and an adjustable effective rigidity. The effective working length (L2) is the distance from the handle distal end (334) to the distal tip (318). It should be understood that probe (300) is substantially similar to probes (100, 200) unless otherwise described herein. Like probes (100, 200), probe (300) has a handle (330), a shaft assembly (310), an adjustment feature (340), and a position sensor (316). The shaft assembly (310) includes an outer tube (312) and an inner shaft (314) like probes (100, 200). The handle (330) has an elongate body (338) and an adjustment feature (340). While not shown, handle (330) may include a grip with an oval section, similar to grip (232) and oval section (233). The elongate body (338) includes a first elongate aperture (344) that optionally has a first aperture detent (346).

The adjustment feature (340) of probe (300) is unlike the adjustment feature (240) of probes (100, 200). The adjustment feature (340) has separate elements for both adjusting the effective working length (L2) and the and the effective rigidity of the shaft assembly (310). The adjustment feature (340) includes a first slider body (342) and a second slider body (343). The first slider body (342) is disposed in a first elongate aperture (344) and includes a second elongate aperture (345), a first slider detent (348), and a second aperture detent (347). The detents (346, 347, 348) are optional. The first slider body (342) is slidably positioned within a first elongate aperture (344) and the first slider body (342) is operatively attached to the outer tube (312). The first slider body (342) is configured to translate the outer tube (312) proximally and distally relative to body (338) and relative to inner shaft (314).

The second slider body (343) is slidably positioned within the second elongate aperture (345). The second elongate aperture (345) has an open distal end (352) and a closed proximal end (354). The open distal end (352) allows the second slider body (343) to remain stationary when the first slider body (342) is translated proximally, as shown in the transition from FIG. 6A to FIG. 6B. The closed proximal end (354) prevents the inner shaft (314) from being transitioned proximally into the outer tube end (313). The second slider body (343) has an optional second slider detent (349). The second slider body (343) is selectively attached to the inner shaft (314) and is configured to selectively translate the inner shaft (314) proximally and distally relative to body (338). In some versions, second slider body (343) is further operable to selectively translate inner shaft relative to outer tube (312). The second slider body (343) engages the inner shaft (314) with an engagement feature (not shown) and translates the inner shaft (314) proximally and distally to change the effective inner shaft length (L3) of the shaft assembly (310). The engagement feature may include a pin, a catch, or some other feature that is configured to engage the inner shaft (314). In some examples, the second slider body (343) is operatively attached to both the inner shaft (314) and the outer tube (312) and is configured to translate both the inner shaft (314) and the outer tube (212) proximally.

The first elongate aperture (344) may include a plurality of first elongate aperture detents (346). In the current example, the first aperture detents (346) and the second aperture detents (347) are arranged at the distal and proximal ends of the first elongate aperture and second elongate aperture (344, 345) respectively. The first and second aperture detents (346, 347) may alternatively be located anywhere along the respective elongate apertures (344, 345) such as at a set interval such as by a distance or by a fractional division. The force that operator applies to the first slider body (342) to overcome the resistance created by the engagement of the first slider detent (348) and the first aperture detent (346) may be greater than the force that the operator applies to overcome the engagement of the second slider detent (349) and the second aperture detent (347) to the translate the second slider body (343) or vice versa. This force differential provides tactile feedback to the operator so that the operator may transition the first slider body (342) independent of the second slider body (343) and the second slider body (343) independent of the first slider body (342).

In another example, the second slider body (343) may be positioned within the first elongate aperture (344) parallel to the first slider body (342). The first slider body (342) is operatively attached to the outer tube (312). The first slider body (342) engages the outer tube (312) to change the effective outer tube length (L1), which in turn changes the effective inner shaft length (L3), which changes the effective rigidity or stiffness of the shaft assembly (310). The outer tube length (L1) is the distance from the handle distal end (334) to the outer tube end (313), and the effective inner shaft length (L3) is the distance from the outer tube end (313) to the distal tip (318).

In the example with a parallel arrangement of slider bodies (342, 343), the first elongate aperture (344) may include a plurality of handle detents (346) arranged on edges of the first elongate aperture (344), to separately engage the first slider body (342) and the second slider body (343). For example, a first slider body (342) may be located on the left-hand side of the first elongate aperture (344), and the second slider body (343) may be located on the right-hand side of the first elongate aperture (344). A first slider body detent (348) may be configured to engage a handle detent (346) located on the left hand side of the first elongate aperture (344) and a second slider body detent (349) may be configured to engage a handle detent (346) located on the right hand side of the first elongate aperture (344). Note that “left” and “right” are merely arbitrary representations and these spatial relationships may be reversed.

FIG. 6A-6B show the adjustment feature (140) with the first slider body (342) transitioning from a distal position (FIG. 6A) to a proximal position (FIG. 6B). The first slider body (342) is configured to adjust the effective rigidity of shaft assembly (310) by changing the effective outer tube length (L1) of the outer tube (312). FIG. 6A shows the probe (300) in a configuration where shaft assembly (310) has a relatively long effective working length (L2) compared to other configurations (see e.g., FIGS. 6C-6D) where shaft assembly (310) is relatively more rigid than shaft assembly (310) is in other configurations (see FIG. 6B). The first slider body (342) in the distal position corresponds to a distal location of the outer tube (312), with a fully extended effective outer tube length (L1). The second slider body (343) is also in the distal position in the state shown in FIG. 6A, and this distal position corresponds to a distal location of the inner shaft (314). The operator engages the first slider body (342) with a thumb or a finger to transition the first slider body (342) proximally to the state shown in FIG. 6B.

FIG. 6B shows the adjustment feature (140) with first slider body (342) fully transitioned to the proximal position while the second slider body (343) remains distally located. The first slider body (342) has engaged the outer tube (312) and has translated the outer tube (312) to the proximal position. The effective outer tube length (L1) is now fully retracted to the proximal position. The effective working length (L2) of the entire shaft assembly (310) remains unchanged because the second slider body (343) was not selectively engaging the inner shaft (314) during the transition from the state shown in FIG. 6A to FIG. 6B, resulting in a shaft assembly (310) that is relatively long compared to other configurations (see FIGS. 6C-6D) and relatively flexible compared to the other configurations (see FIGS. 6A and 6C-6D).

FIG. 6C shows the first slider body (342) in the distal position and the second slider body (343) proximally located within the second elongate aperture (345). The second slider body (343) abuts the closed proximal end (354). The closed proximal end (354) prevents the second slider body (343) from transitioning the inner shaft (314) proximally into the outer tube end (313). In FIG. 6A, the first slider body (342) was in the distal position and the second slider body (343) was in the distal position. To achieve the state shown in FIG. 6C, the operator presses the second slider body (343) with a thumb or a finger to engage the inner shaft (314). The operator transitions the second slider body (343) from the distal position (FIG. 6A) proximally relative to the second elongate aperture (345) with a thumb or finger while holding the first slider body (342) stationary with an additional finger. The second slider body (343) engages the inner shaft (314) with the engagement feature (not shown) to translate the inner shaft (314) proximally. The inner shaft (314) being translated proximally reduces the effective inner shaft length (L3), which in turn decreases the effective working length (L2). The reduction of the effective inner shaft length (L3) increases the rigidity of the shaft assembly (310), resulting in a shaft assembly (310) that is relatively short compared to other configurations (FIGS. 6A-6B) and relatively rigid compared to the other configurations (see FIG. 6A-6B).

In order to retain the same effective rigidity of shaft assembly (310), the same effective inner shaft length (L3) must be maintained. This same rigidity is maintained by a coordinated proximal movement of both the first slider body (342) and the second slider body (343). FIG. 6D shows the first slider body (342) and the second slider body (343) after a coordinated proximal movement of both the first slider body (342) and the second slider body (343). The second slider body (343) is located in a most proximal position relative to both elongate apertures (344, 345). In FIG. 6C the first slider body (342) is in the distal position and the second slider body (343) is in the proximal position relative to the second elongate aperture (345). The operator transitions the first and second slider bodies (342, 343) from the distal position to the proximal position in FIG. 6D. The operator may engage both the first and second slider bodies (342, 343) with a finger or thumb and transition the first and second slider bodies (342, 343) proximally. Additionally, the operator may engage the second slider body (343) with a finger or thumb and the second slider body (343) may engage the closed proximal end (354) thereby transitioning the first slider body (342) proximally.

The first slider body (342) engages the outer tube (312) and translates the outer tube (312) proximally. The second slider body (343) engages the inner tube (314) and translates the inner tube (314) proximally. The effective outer tube length (L1) is reduced. The effective working length (L2) is reduced, and the effective inner shaft length (L3) remains constant, resulting in a shaft assembly (310) that is relatively short compared to other configuration (see FIGS. 6A-6C) and relatively rigid compared to the other configurations (see FIG. 6A-6B).

V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a handle including an elongate body; (b) an elongate shaft assembly, the elongate shaft assembly including: (i) an outer tube slidably disposed within the elongate body, the outer tube extending distally from the elongate body to an outer tube distal end, and (ii) an inner shaft coaxially disposed within the outer tube, the inner shaft extending distally through the outer tube distal end to an inner shaft distal tip, the inner shaft being sized and configured to pass through passageways within an ear, nose, or throat of a patient; (c) an adjustment feature slidably disposed relative to the elongate body of the handle and configured to translate at least the outer tube relative to the handle and thereby adjust one or more characteristics of the elongate shaft assembly; and (d) a position sensor configured to generate signals indicating a real-time position of the inner shaft distal tip.

### Example 2

The apparatus of Example 1, wherein the outer tube is rigid, and the inner shaft is flexible.

### Example 3

The apparatus of Example 2, wherein the one or more characteristics includes a rigidity of the elongate shaft assembly, wherein the rigidity is adjustable by a linear movement of the adjustment feature relative to the elongate body of the handle.

### Example 4

The apparatus of Example 2, wherein the one or more characteristics includes an effective length of the elongate shaft assembly, wherein the effective length is adjustable with a linear motion of the adjustment feature relative to the elongate body of the handle.

### Example 5

The apparatus of Example 2, wherein the one or more characteristics includes an effective length of the elongate shaft assembly and a rigidity of the elongate shaft assembly, wherein the characteristics are adjustable by a linear motion of the adjustment feature relative to the elongate body of the handle.

### Example 6

The apparatus of Example 5, wherein the adjustment feature is operable to adjust an effective length of the outer tube, wherein the adjustment feature is further operable to adjust the effective length of the elongate shaft assembly.

### Example 7

The apparatus of any one or more of Examples 1 through 6, wherein the adjustment feature includes: (i) a first slider body configured to adjust an effective length of the outer tube, and (ii) a second slider body configured to adjust an effective length of the inner shaft.

### Example 8

The apparatus of Example 7, wherein the first slider body and the second slider body are disposed within a first elongate aperture defined by the elongate body of the handle.

### Example 9

The apparatus of Example 7, wherein the first slider body includes a second elongate aperture, and the second slider body is disposed within the second elongate aperture.

### Example 10

The apparatus of Example 9, wherein the second slider body includes a detent, wherein the second elongate aperture includes a plurality of second aperture detents arranged along the second elongate aperture, wherein the second aperture detents are configured to engage the detent of the second slider body to resist movement of the second slider body along the second elongate aperture.

### Example 11

The apparatus of Example 10, wherein the second aperture detents are spaced apart from each other at intervals corresponding to three or more discrete longitudinal positions of the inner shaft relative to the elongate body of the handle.

### Example 12

The apparatus of any one or more of Examples 1 through 6, wherein the adjustment feature includes a first slider body slidably disposed in a first elongate aperture of the handle, wherein the first slider body includes a detent, wherein the first elongate aperture includes a plurality of first aperture detents arranged along the first elongate aperture, wherein the first aperture detents are configured to engage the detent of the first slider body to resist movement of the first slider body along the first elongate aperture.

### Example 13

The apparatus of Example 12, wherein the first aperture detents are spaced apart from each other at intervals corresponding to three or more discrete longitudinal positions of the outer tube relative to the elongate body of the handle.

### Example 14

The apparatus of any one or more of Examples 1 through 13, wherein the outer tube is also sized and configured to pass through passageways within an ear, nose, or throat of a patient.

### Example 15

A system comprising: (a) an image guided navigation system including: (i) a processor, and (ii) a plurality of magnetic field generators configured to generate a magnetic field; and (b) a probe, wherein the probe includes: (i) a handle; (ii) a shaft assembly extending distally from the handle, the shaft assembly including: (A) a rigid outer tube having a distal end, and (B) a flexible inner shaft having a distal tip, the distal tip of the flexible inner shaft being positionable distally of the distal end of the rigid outer tube; (iii) an adjustment feature configured to adjust a longitudinal position of at least the rigid outer tube relative to the handle; and (iv) a position sensor in communication with the processor, wherein the position sensor is configured to generate a signal in response to the magnetic field generated by the plurality of magnetic field generators, wherein the processor is configured to utilize the signal to determine a position of the flexible distal tip of the inner shaft in three-dimensional space.

### Example 16

The system of Example 15, wherein the shaft assembly has a rigidity that is configured to change based on the longitudinal position of at least the rigid outer tube relative to the handle.

### Example 17

The system of any one or more of Examples 15 through 16, wherein the shaft assembly has an effective length that is configured to change based on the longitudinal position of at least the rigid outer tube relative to the handle.

### Example 18

The system of Example 15, wherein the shaft assembly has a rigidity and an effective length that are each configured to change based on the longitudinal position of at least the rigid outer tube relative to the handle.

### Example 19

The system of Example 16, wherein the handle includes an elongate aperture, wherein the adjustment feature is disposed within the elongate aperture and the adjustment feature is configured to adjust the rigidity of the shaft assembly by a linear translation of the adjustment feature relative to the handle.

### Example 20

The system of any one or more of Examples 15 through 19, wherein the adjustment feature includes: (A) a first slider operable to adjust the longitudinal position of the rigid outer tube relative to the handle, and (B) a second slider operable to adjust the longitudinal position of the flexible inner shaft relative to the handle.

### Example 21

An apparatus, comprising: (a) a handle that includes a first elongate aperture; (b) an elongate shaft assembly extending distally from the handle, the elongate shaft assembly including: (i) a rigid outer shaft having a distal end, and (ii) a flexible inner shaft having a distal tip, the distal tip of the flexible inner shaft being positionable distally of the distal end of the rigid outer shaft; (c) an adjustment feature including: (i) a first slider body operable to translate relative to the handle, the first slider body being operable to drive the rigid outer shaft to translate relative to the handle, and (ii) a second slider body operable to translate relative to the handle, the second slider body being operable to drive at least the flexible inner shaft to translate relative to the handle; and (d) a position sensor configured to generate signals indicating a real-time position of the distal tip of the flexible inner shaft.

VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, or steam.

Having shown and described various versions of the present invention, further adaptations of the methods and systems described herein may be accomplished by appropriate modifications by one skilled in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. An apparatus, comprising:
(a) a handle including an elongate body;
(b) an elongate shaft assembly, the elongate shaft assembly including:
(i) an outer tube slidably disposed within the elongate body, the outer tube extending distally from the elongate body to an outer tube distal end, and
(ii) an inner shaft coaxially disposed within the outer tube, the inner shaft extending distally through the outer tube distal end to an inner shaft distal tip, the inner shaft being sized and configured to pass through passageways within an ear, nose, or throat of a patient;
(c) an adjustment feature slidably disposed relative to the elongate body of the handle and configured to translate at least the outer tube relative to the handle and thereby adjust one or more characteristics of the elongate shaft assembly; and
(d) a position sensor configured to generate signals indicating a real-time position of the inner shaft distal tip.

2. The apparatus of claim 1, wherein the outer tube is rigid, and the inner shaft is flexible.

3. The apparatus of claim 2, wherein the one or more characteristics includes a rigidity of the elongate shaft assembly, wherein the rigidity is adjustable by a linear movement of the adjustment feature relative to the elongate body of the handle.

4. The apparatus of claim 2, wherein the one or more characteristics includes an effective length of the elongate shaft assembly, wherein the effective length is adjustable with a linear motion of the adjustment feature relative to the elongate body of the handle.

5. The apparatus of claim 2, wherein the one or more characteristics includes an effective length of the elongate shaft assembly and a rigidity of the elongate shaft assembly, wherein the characteristics are adjustable by a linear motion of the adjustment feature relative to the elongate body of the handle.

6. The apparatus of claim 5, wherein the adjustment feature is operable to adjust an effective length of the outer tube, wherein the adjustment feature is further operable to adjust the effective length of the elongate shaft assembly.

7. The apparatus of claim 1, wherein the adjustment feature includes:
(i) a first slider body configured to adjust an effective length of the outer tube, and
(ii) a second slider body configured to adjust an effective length of the inner shaft.

8. The apparatus of claim 7, wherein the first slider body and the second slider body are disposed within a first elongate aperture defined by the elongate body of the handle.

9. The apparatus of claim 7, wherein the first slider body includes a second elongate aperture, and the second slider body is disposed within the second elongate aperture.

10. The apparatus of claim 9, wherein the second slider body includes a detent, wherein the second elongate aperture includes a plurality of second aperture detents arranged along the second elongate aperture, wherein the second aperture detents are configured to engage the detent of the second slider body to resist movement of the second slider body along the second elongate aperture.

11. The apparatus of claim 10, wherein the second aperture detents are spaced apart from each other at intervals corresponding to three or more discrete longitudinal positions of the inner shaft relative to the elongate body of the handle.

12. The apparatus of claim 1, wherein the adjustment feature includes a first slider body slidably disposed in a first elongate aperture of the handle, wherein the first slider body includes a detent, wherein the first elongate aperture includes a plurality of first aperture detents arranged along the first elongate aperture, wherein the first aperture detents are configured to engage the detent of the first slider body to resist movement of the first slider body along the first elongate aperture.

13. The apparatus of claim 12, wherein the first aperture detents are spaced apart from each other at intervals corresponding to three or more discrete longitudinal positions of the outer tube relative to the elongate body of the handle.

14. A system comprising:
(a) an image guided navigation system including:
(i) a processor, and
(ii) a plurality of magnetic field generators configured to generate a magnetic field; and
(b) a probe, wherein the probe includes:
(i) a handle;
(ii) a shaft assembly extending distally from the handle, the shaft assembly including:
(A) a rigid outer tube having a distal end, and
(B) a flexible inner shaft having a distal tip, the distal tip of the flexible inner shaft being positionable distally of the distal end of the rigid outer tube;
(iii) an adjustment feature configured to adjust a longitudinal position of at least the rigid outer tube relative to the handle; and
(iv) a position sensor in communication with the processor, wherein the position sensor is configured to generate a signal in response to the magnetic field generated by the plurality of magnetic field generators, wherein the processor is configured to utilize the signal to determine a position of the distal tip of the flexible inner shaft in three-dimensional space.

15. The system of claim 14, wherein the shaft assembly has a rigidity that is configured to change based on the longitudinal position of at least the rigid outer tube relative to the handle.

16. The system of claim 14, wherein the shaft assembly has an effective length that is configured to change based on the longitudinal position of at least the rigid outer tube relative to the handle.

17. The system of claim 14, wherein the shaft assembly has a rigidity and an effective length that are each configured to change based on the longitudinal position of at least the rigid outer tube relative to the handle.

18. The system of claim 15, wherein the handle includes an elongate aperture, wherein the adjustment feature is disposed within the elongate aperture and the adjustment feature is configured to adjust the rigidity of the shaft assembly by a linear translation of the adjustment feature relative to the handle.

19. The system of claim 14, wherein the adjustment feature includes:
(A) a first slider operable to adjust the longitudinal position of the rigid outer tube relative to the handle, and
(B) a second slider operable to adjust the longitudinal position of the flexible inner shaft relative to the handle.

20. An apparatus, comprising:
(a) a handle that includes a first elongate aperture;
(b) an elongate shaft assembly extending distally from the handle, the elongate shaft assembly including:
(i) a rigid outer shaft having a distal end, and
(ii) a flexible inner shaft having a distal tip, the distal tip of the flexible inner shaft being positionable distally of the distal end of the rigid outer shaft;
(c) an adjustment feature including:
(i) a first slider body operable to translate relative to the handle, the first slider body being operable to drive the rigid outer shaft to translate relative to the handle, and
(ii) a second slider body operable to translate relative to the handle, the second slider body being operable to drive at least the flexible inner shaft to translate relative to the handle; and
(d) a position sensor configured to generate signals indicating a real-time position of the distal tip of the flexible inner shaft.
